(19) United States
(12) Patent Application Publication
(10) Pub. No.: US 2010/0226933 A1
(43) Pub. Date: Sep. 9, 2010

(54) HYPOALLERGENIC PROTEINS

(75) Inventors: Josef Thalhamer, Lamprechtshausen (AT); Arnulf Hartl, Anif (AT); Manfred Sippl, Oberndorf (AT); Peter Lackner, Salzburg (AT); Fatima Ferreira-Briza, Salzburg (AT); Theresa Thalhamer, Lamprechtshausen (AT); Hanno Stutz, Freilassing (DE)

Correspondence Address: JOYCE VON NATZMER;PEQUIGNOT + MYERS LLC, 200 Madison Avenue, Suite 1901, New York, NY, 10016, US

(73) Assignee: UNIVERSITAET SALZBURG, Salzburg (AT)

(21) Appl. No.: 12/526,267

(22) PCT Filed: Feb. 13, 2008
(86) PCT No.: PCT/AT2008/000049

§ 371 Date: Aug. 7, 2009

(30) Foreign Application Priority Data
Feb. 14, 2007 (AT) A 231/2007
Feb. 20, 2007 (AT) A 261/2007

(51) Int. Cl. A61K 39/00; G01N 33/53; G01N 33/00; C07K 14/00; A61P 37/08; G06F 19/00

(52) U.S. Cl. 424/185.1; 435/7.1; 435/7.21; 436/86; 530/350; 702/19

(57) ABSTRACT

The present invention relates to a method for identifying a hypoallergenic derivative of a wild-type allergen comprising: determining the three-dimensional structure of a wild-type allergen; introducing at least one point mutation into said wild-type allergen, thereby obtaining a mutated allergen; identifying a hypoallergenic derivative of said wild-type allergen by detecting a destabilisation or change of the three-dimensional structure of the mutated allergen compared to the wild-type allergen by determining energy differences between the wild-type allergen and the mutant allergen expressed as a Z-score.

The present invention relates to a method for identifying hypoallergenic molecules.

Type I allergy has become a major health issue in developed countries. Up to 20% of the population suffers from allergies making it not only a health but also an economical problem. So far specific immunotherapy (SIT) has been the only approved curative treatment available. But SIT still harbours some major drawbacks such as severe side effects (e.g. anaphylactic shock) that can occur due to application of high doses of allergen. Another disadvantage of the current protocol is the use of allergen extracts containing other potentially allergenic proteins leading to new sensitisation events during the treatment.

Since many allergens have become available as recombinant molecules, few representative allergens covering most of the IgE epitopes could be selected for diagnostic purpose to establish a molecule-based diagnosis. This would pave the way for the use of both, a component-resolved diagnosis as well as a custom-made therapy for type I allergy. Nevertheless, recombinant native allergens still have the capacity to crosslink pre-existing IgE and can thus trigger anaphylactic side effects.

In the past years this problem has been addressed and various approaches have been published to produce allergen derivatives with reduced IgE binding (hypoallergens) while preserving sequence and structural motifs necessary for T cell recognition (T cell epitopes) and to induce IgG antibodies reactive with the natural allergen (blocking antibodies).

In principle, the majority of studies dealing with hypoallergenic allergen derivatives used two approaches, i.e. fragmentation/fusion and the insertion of mutations. The former mainly intended to destroy the native structure (at least in parts) of the molecule, whereas concerning the latter approach, natural hypoallergens served as a template for the mutations.

Recombinant allergens fulfill the requirements necessary for this strategy because they enable a molecular diagnosis and therapy, and their production and purification in large scale is feasible. Therefore, recombinant proteins will stepwise replace allergen extracts for SIT in the near future. However, two events responsible for side effects with allergen extracts, are still valid for recombinant allergens, i.e. crosslinking of pre-existing IgE in the allergic individual and the possible induction of new IgE antibodies triggered by the therapeutic pro tocol. These aspects have led to an intensive investigation of natural and artificial hypoallergens which are primarily defined by the lack of IgE-binding measured with sera from allergic patients.

In the past decade, numerous attempt has been made to find or develop hypoallergenic molecules which could be used for SIT with recombinant allergens. Investigation of the IgE-binding activity of isoallergens led to the identification of naturally occurring hypoallergens, such as Bet v 1 hypoallergens such as Bet v 1d, Bet v 1 g and Bet v 11.

In Buhot et al. (Portein Sci. 13 (2004): 2970-2978) mutants comprising over 20 single mutations and a deletion of 10 amino acid residues of the allergen Api m 1 are described. The overall structure of said mutants created by combining several point mutations is more or less similar to the wild type Api m 1.

Westritschnig et al. (J. Immunol. 72 (2004): 5684-5692) describe a modified Phl p 7 molecule which has been modified in order to destroy the region of the wild type Phl p 7 which is responsible for its calcium binding property.

In Neudecker et al. (Biochem. J. 376 (2003): 97-107) cross-reactive IgE epitopes of Pru av 1 and Api g 1 and mutants derived from said allergens are disclosed. However, the three-dimensional structure (determined as Z-score) of the mutants do not significantly differ from the wild type allergens.

Verdino et al. (EMBO J. 21 (19) (2002): 5007-5016) describe the structure of the allergen Phl p 7 and possibilities to create hypoallergenic variants.

A review article of Valenta et al. (Curr. Opin. Immunol. 14 (2002): 718-727) discusses allergen derivatives which can be used in allergen specific immunotherapy. This article emphasises that the relevant T cell epitopes of the allergen derivatives have to be conserved in the derivatives in order to induce respective protective antibody response.

It is an object of the present invention to provide a new method for the identification and manufacture of hypoallergenic molecules derived from wild-type allergens. Conventional methods for the identification of possible hypoallergenic molecules usually require complex and laborious steps, because the hypoallergenicity of modified allergens has always to be proven in many in vitro as well as in vivo experiments.

Therefore, the present invention relates to a method for identifying a hypoallergenic derivative of a wild-type allergen comprising:
determining the three-dimensional structure of a wild-type allergen,
introducing at least one point mutation into said wild-type allergen thereby obtaining a mutated allergen molecule,
optionally determining the three-dimensional structure of said mutated allergen molecule,
identifying a hypoallergenic derivative of said wild-type allergen by detecting a destabilisation or change of the three-dimensional structure of the mutated allergen compared to the wild-type allergen by determining energy differences between the wild-type allergen and the mutant allergen expressed as a Z-score. A preferred procedure for deriving destabilizing multimutant sequences is: (i) Calculate all single point mutations. (ii) Take the mutant with highest increase in the combined Z-score. (iii) Use this mutant sequence and repeat step (i) until a mutant with a Z-score increase of a predefined minimal value (6%, e.g., 8%, 10%, 12%, 15%), compared to wild type appears. This mutants show significant changes in the three-dimensional structure as shown, for instance, in Table 3 and which is a prerequisite for a hypoallergen (compared to the wild type allergen).

It turned out that the difference between the Z-scores of a wild-type allergen and a mutated allergen molecule was a reliable indicator as to whether the mutated allergen molecule is effectively hypoallergenic. An allergen or allergenic molecule can be considered as being “hypoallergenic” when the Z-score of the mutated allergen or allergenic molecule is increased for about at least 5%, preferably for about at least 6%, more preferably for about at least 7%, even more preferably for about at least 8%, most preferred for about at least 10%.

The Z-score of a protein is defined as the energy separation between the native fold and the average of an ensemble of misfolds in the units of the standard deviation of the ensemble. The Z-score is often used as a way of testing the knowledge-based potentials for their ability to recognise the native fold from other alternatives. In protein folding studies, knowledge-based potentials derived from a statistical analysis of known protein structures (Sippl M J Current Opinion in Structural Biology (1995) 5:229-235) are frequently used in simplified models of proteins. The quality of such potentials is often assessed by so-called Z-scores, which test how well the potentials differentiate the native fold of a protein from an ensemble of misfolded structures. The Z-score expresses, to what degree a certain protein sequence fits a certain three-dimensional structure, normalized by a random background model. Given a protein sequence S and any structure X, an energy E(S,X) may be calculated. Using a large number of randomly selected three-dimensional structures Xi, the distribution of energy values E(S,Xi) can be calculated and hence its mean Em and the standard deviation sigma. Then, the Z-score of a particular sequence-structure pair E(S,P) is defined by Z-score=(E(S,P)-Em)/sigma. In case P is the wild type structure and S is the wild type sequence the Z-score is derived from the wild type protein. Consequently, if a change in the wild type sequence is introduced and the procedure for mutated sequence and wild type structure is repeated, the influence of the mutation on the protein structure can be determined. A increase in Z-score indicates that the mutated sequence fits less well to the wild type structure and thus has a destabilizing effect. A decrease in Z-score indicates a stabilizing effect for the three-dimensional structure. Destabilization, however, does not necessarily prevent the mutated protein to undergo protein folding, but at least means, that it is expected to undergo a conformational change probably resulting in a new stable conformation. Three types of Z-scores regarding the kind of physical interaction investigated are distinguished. The pair Z-score describes the energies resulting from interactions between pairs of aminoacids within the protein molecule and the surface Z-score describes the interactions between the protein and the surrounding solvent. Combined Z-scores are derived from a linear combination of the two energy distributions and are thus a global indicator of protein stability.

A preferred procedure for deriving destabilizing multimutant sequences is: (i) Calculate all single point mutations. (ii) Take the mutant with highest increase in the combined Z-score. (iii) Use this mutant sequence and repeat step (i) until a mutant with a Z-score increase of a predefined minimal value compared to the wild type appears. This mutants show significant changes in the three-dimensional structure as shown in Table 3 and which is a prerequisite for a hypoallergen (compared to the wild type allergen).

“Derivative”, as used herein, refers to peptides, polypeptides and proteins, which are derived from a wild-type molecule. “Derivatives” comprise modifications (at least one point mutation, chemical modifications of at least one amino acid residue etc.) in their amino acid sequence compared to a wild-type protein, in particular compared to a wild-type allergen.

The term “at least one point mutation”, as used herein refers to a type of mutation resulting from a single amino acid substitution, whereby a wild-type allergen may comprise one, two, three, four, five, ten or even more point mutations. However, it is preferred that the mutated hypoallerene derivative comprises a low number of point mutations in order to substantially preserve T cell epitopes of the wild-type allergen in the derivative.

These Z-scores may be calculated by the method of Bowie et al. (Science (1991) 253:164-170) and Sippl (J Comput Aided Mol Des (1993) 7:473-501).

According to a preferred embodiment of the present invention the three-dimensional structure is determined by a method selected from the group consisting of nuclear magnetic resonance (NMR) spectroscopy, X-ray crystallography, computational methods, circular dichroism and combinations thereof.

The data obtained from the three dimensional structure of a molecule are used for the determination of the Z-score as described above. The methods mentioned above are regularly used to determine the three-dimensional structure and are therefore well known to the person skilled in the art.

In order to evaluate the IgE binding capacity of the mutated allergen, said mutated allergen is further subjected to an IgE binding assay or a mediator release assay, wherein the IgE binding assay is preferably a RIST (radio immunosorbens test), a RAST (radio allergo-sorbens test) or a Western blot.

IgE binding assays may be performed as known in the art and similar to a Western Blot by using allergens or the mutated allergens immobilised on a surface, preferably a membrane, and contacted with an IgE comprising sample. For instance, suitable membranes may be cellulose membranes on which allergens or mutated allergens are immobilised by washing the membrane with Tris-buffered saline (TBS) and then incubating the membrane with blocking solution overnight at room temperature. After blocking, the membranes are incubated with serum from patients with allergen hypersensitivity diluted (1:5) in a solution containing TBS and 1% bovine serum albumin for at least 12 h at 4° C. or 2 h at room temperature. Detection of the primary antibody can be performed with ¹²⁵I-labelled anti-IgE antibody.

The RIST test measures the total IgE. A paper disc, for instance, to which an anti-IgE has been bound, is incubated with a drop of the patient's serum. This disc binds all of the IgE in the sample. The disc is then washed to remove extraneous materials and radioactively labelled anti-IgE is added for a second incubation. During this time, the labelled anti-IgE reacts with IgE molecules previously bound to the disc and after a final washing step, the amount of radioactivity bound to the disc is measured in a gammacounter. The amount of radioactivity binding the test serum is then compared to the binding obtained by serial dilutions of a RIST reference standard known to contain exactly 100 units of IgE. The use of a suitable reference standard is a basic requirement for all radioimmunoassay determinations, because there may be unexpected variables due to changes in incubation time, changes in room temperature, and decay in the amount of radioactivity bound to the anti-IgE used in the second stage.

The RAST test is a measurement of a specific allergen. The allergen of interest, such as short ragweed or birch pollen allergen, is bound to a disc and reacts only with the allergen IgE in the sample. After the initial incubation, non-specific IgE antibody and other proteins are removed by washing. Radioactively-labelled anti-IgE is then added and allowed to incubate overnight thereby forming a radioactive complex with the specific IgE. The radioactivity is then compared to a standard.

The mediator release assays to be used in the method of the present invention are based on the principle that IgE antibodies bound to receptors on the basophil membrane surface are cross-linked by allergen molecules or anti-IgE antibody, whereby this stimulation causes a degranulation reaction, resulting in chemical mediator release. This corresponds to chemical mediator release by allergic reaction (IgE-mediated specific chemical mediator release). In another type of chemical mediator release, chemical mediator release occurs directly without crosslinking of the IgEs on the basophil membrane surface. In contrast to the IgE-mediated specific chemical mediator release, this second type of chemical mediator release can occur even in the absence of anti-IgE antibody and allergen (non-specific chemical mediat- or release).

According to a preferred embodiment of the present invention the mediator release assay is a CAST (cellular allergen stimulation test), a histamine release assay, a leukotriene C4 release assay, a cysteinyl leukotriene release assay, tryptase assay, or rat basophil leukemia (RBL) cell release assay.

In the diagnosis and pathologic analysis of allergic diseases, it is useful to test IgE-mediated specific chemical mediator release, typically by the histamine release test. Histamine, a very important chemical mediator causing type I allergic reactions, is known to induce various reactions such as bronchial smooth muscle constriction and accentuating of vascular permeability. The histamine release test is a unique testing method based on a biological reaction, in which immunoglobulin E (IgE) bound via receptor onto the human peripheral blood basophil surface is reacted with allergen or hypoallergenic molecules to release histamine. The amount of histamine released by this test is determined.

This histamine release test using peripheral blood can be carried out in two different ways, either by using whole blood or by using washed leukocytes. Although the whole blood method may be useful in generally determining the patient's allergic condition, there is the possibility that non-basophil serum components affect histamine release assay. For this reason, it is common practice to use washed leukocytes when accurate basophil reactivity is analysed for research into the mechanism of action of drugs etc. or for basic research into the mechanism of histamine release. However, separation of washed leukocytes requires troublesome procedures, including erythrocyte removal with dextran solution, followed by two or three cycles of centrifugation and washing and subsequent leukocyte count adjustment. This results in a requirement for an increased volume of blood for the test. These drawbacks pose many problems for the use of the washed leukocyte method as a routine testing method.

The wild-type allergen to be modified is preferably selected from the group consisting of Amb a 1, Amb a 2, Amb a 3, Amb a 5, Amb a 6, Amb a 7, Amb a 8, Amb a 9, Amb a 10, Amb t 5, Art v 1, Art v 2, Art v 3, Art v 4, Art v 5, Art v 6, Hel a 1, Hel a 2, Hel a 3, Mer a 1, Che a 1, Che a 2, Che a 3, Sal k 1, Cat r 1, Pla l 1, Hum j 1, Par j 1, Par j 2, Par j 3, Par o 1, Cyn d 1, Cyn d 7, Cyn d 12, Cyn d 15, Cyn d 22w, Cyn d 23, Cyn d 24, Dac g 1, Dac g 2, Dac g 3, Dac g 5, Fes p 4w, Hol l 1, Lol p 1, Lol p 2, Lol p 3, Lol p 5, Lol p 11, Pha a 1, Phl p 1, Phl p 2, Phl p 4, Phl p 5, Phl p 6, Phl p 11, Phl p 12, Phl p 13, Poa p 1, Poa p 5, Sor h 1, Pho d 2, Aln g 1, Bet v 1, Bet v 2, Bet v 3, Bet v 4, Bet v 6, Bet v 7, Car b 1, Cas s 1, Cas s 5, Cas s 8, Cor a 1, Cor a 2, Cor a 8, Cor a 9, Cor a 10, Cor a 11, Que a 1, Fra e 1, Lig v 1, Ole e 1, Ole e 2, Ole e 3, Ole e 4, Ole e 5, Ole e 6, Ole e 7, Ole e 8, Ole e 9, Ole e 10, Syr v 1, Cry j 1, Cry j 2, Cup a 1, Cup s 1, Cup s 3w, Jun a 1, Jun a 2, Jun a 3, Jun o 4, Jun s 1, Jun v 1, Pla a 1, Pla a 2, Pla a 3, Aca s 13, Blo t 1, Blot 3, Blo t 4, Blo t 5, Blo t 6, Blo t 10, Blo t 11, Blo t 12, Blo t 13, Blo t 19, Der f 1, Der f 2, Der f 3, Der f 7, Der f 10, Der f 11, Der f 14, Der f 15, Der f 16, Der f 17, Der f 18w, Der m 1, Der p 1, Der p 2, Der p 3, Der p 4, Der p 5, Der p 6, Der p 7, Der p 8, Der p 9, Der p 10, Der p 11, Der p 14, Der p 20, Der p 21, Eur m 2, Eur m 14, Gly d 2, Lep d 1, Lep d 2, Lep d 5, Lep d 7, Lep d 10, Lep d. 13, Tyr p 2, Tyr p 13, Bos d 2, Bos d 3, Bos d 4, Bos d 5, Bos d 6, Bos d 7, Bos d 8, Can f 1, Can f 2, Can f 3, Can f 4, Equ c 1, Equ c 2, Equ c 3, Equ c 4, Equ c 5, Fel d 1, Fel d 2, Fel d 3, Fel d 4, Fel d 5w, Fel d 6w, Fel d 7w, Cav p 1, Cav p 2, Mus m 1, Rat n 1, Alt a 1, Alt a 3, Alt a 4, Alt a 5, Alt a 6, Alt a 7, Alt a 8, Alt a 10, Alt a 12, Alt a 13, Cla h 2, Cla h 5, Cla h 6, Cla h 7, Cla h 8, Cla h 9, Cla h 10, Cla h 12, Asp fl 13, Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 15, Asp f 16, Asp f 17, Asp f 18, Asp f 22w, Asp f 23, Asp f 27, Asp f 28, Asp f 29, Asp n 14, Asp n 18, Asp n 25, Asp o 13, Asp o 21, Pen b 13, Pen b 26, Pen ch 13, Pen ch 18, Pen ch 20, Pen c 3, Pen c 13, Pen c 19, Pen c 22w, Pen c 24, Pen o 18, Fus c 1, Fus c 2, Tri r 2, Tri r 4, Tri t 1, Tri t 4, Cand a 1, Cand a 3, Cand b 2, Psi c 1, Psi c 2, Cop c 1, Cop c 2, Cop c 3, Cop c 5, Cop c 7, Rho m 1, Rho m 2, Mala f 2, Mala f 3, Mala f 4, Mala s 1, Mala s 5, Mala s 6, Mala s 7, Mala s 8, Mala s 9, Mala s 10, Mala s 11, Mala s 12, Mala s 13, Epi p 1, Aed a 1, Aed a 2, Api m 1, Api m 2, Api m 4, Api m 6, Api m 7, Bom p 1, Bom p 4, Bla g 1, Bla g 2, Bla g 4, Bla g 5, Bla g 6, Bla g 7, Bla g 8, Per a 1, Per a 3, Per a 6, Per a 7, Chi k 10, Chi t 1-9, Chi t 1.01, Chi t 1.02, Chi t 2.0101, Chi t 2.0102, Chi t 3, Chi t 4, Chi t 5, Chi t 6.01, Chi t 6.02, Chi t 7, Chi t 8, Chi t 9, Cte f 1, Cte f 2, Cte f 3, Tha p 1, Lep s 1, Dol m 1, Dol m 2, Dol m 5, Dol a 5, Pol a 1, Pol a 2, Pol a 5, Pol d 1, Pal d 4, Pol d 5, Pol e 1, Pol e 5, Pol f 5, Pol g 5, Pol m 5, Vesp c 1, Vesp c 5, Vesp m 1, Vesp m 5, Ves f 5, Ves g 5, Ves m 1, Ves m 2, Ves m 5, Ves p 5, Ves s 5, Ves vi 5, Ves v 1, Ves v 2, Ves v 5, Myr p 1, Myr p 2, Sol g 2, Sol g 4, Sol i 2, Sol i 3, Sol i 4, Sol s 2, Tria p 1, Gad c 1, Sal s 1, Bos d 4, Bos d 5, Bos d 6, Bos d 7, Bos d 8, Gal d 1, Gal d 2, Gal d 3, Gal d 4, Gal d 5, Met e 1, Pen a 1, Pen i 1, Pen m 1, Pen m 2, Tod p 1, Hel as 1, Hal m 1, Ran e 1, Ran e 2, Bra j 1, Bra n 1, Bra o 3, Bra r 1, Bra r 2, Hor v 15, Hor v 16, Hor v 17, Hor v 21, Sec c 20, Tri a 18, Tri a 19, Tri a 25, Tri a 26, Zea m 14, Zea m 25, Ory s 1, Api g 1, Api g 4, Api g 5, Dau c 1, Dau c 4, Cor a 1.04, Car a 2, Car a 8, Fra a 3, Fra a 4, Mal d 1, Mal d 2, Mal d 3, Mal d 4, Pyr c 1, Pyr c 4, Pyr c 5, Pers a 1, Pru ar 1, Pru ar 3, Pru av 1, Pru av 2, Pru av 3, Pru av 4, Pru d 3, Pru du 4, Pru p 3, Pru p 4, Aspa 1, Cro s 1, Cro s 2, Lac s 1, Vit v 1, Mus xp 1, Ana c 1, Ana c 2, Cit l 3, Cit s 1, Cit s 2, Cit s 3, Lit c 1, Sin a 1, Gly m 1, Gly m 2, Gly m 3, Gly m 4, Vig r 1, Ara h 1, Ara h 2, Ara h 3, Ara h 4, Ara h 5, Ara h 6, Ara h 7, Ara h 8, Len c 1, Len c 2, P is s 1, P is s 2, Act c 1, Act c 2, Cap a 1w, Cap a 2, Lyc e 1, Lyc e 2, Lyc e 3, Sola t 1, Sola t 2, Sola t 3, Sola t 4, Ber e 1, Ber e 2, Jug n 1, Jug n 2, Jug r 1, Jug r 2, Jug r 3, Ana o 1, Ana o 2, Ana o 3, Rlc c 1, Ses i 1, Ses i 2, Ses i 3, Ses i 4, Ses i 5, Ses i 6, Cuc m 1, Cuc m 2, Cuc m 3, Ziz m 1, Ani s 1, Ani s 2, Ani s 3, Ani s 4, Arg r, Asc s 1, Car p 1, Den n 1, Hev b 1, Hev b 2, Hev b 3, Hev b 4, Hev b 5, Hev b 6.01, Hev b 6.02, Hev b 6.03, Hev b 7.01, Hev b 7.02, Hev b 8, Hev b 9, Hev b 10, Hev b 11, Hev b 12, Hev b 13, Hom s 1, Hom s 2, Hom s 3, Hom s 4, Hom s 5 and Trip s 1, wherein it is especially preferred to modify those allergens which are responsible for the most allergic conditions. Particularly preferred allergens are selected from the group consisting of Amb a 1, Art v 1, Par j 1, Cyn d 1, Dac g 1, Lol p 1, Phl p 1, Phl p 2, Phl p 4, Phl p 5, Phl p 6, Aln g 1, Bet v 1, Cas s 1, Cor a 1, Que a 1, Ole e 1, Cry j 1, Jun a 1, Der f 1, Der m 1, Der p 1, Equ c 1, Fel d 1, Alt a 1, Cla h 2, Asp f 1, Pen b 13, Cand a 1, Api m 1, Pol a 1, Vesp c 1, Bra j 1, Bra n 1, Bra o 3, Bra r 1, Zea m 14, Api g 1, Dau c 1, Mal d 1, Pru ar 1, Pru av 1, Ara h 1, Cap a 1w, Lyc e 1, Hev b 1, Hev b 2, Hev b 3, Hev b 4, Hev b 5, Hev b 6 and Hom 1.

According to the present invention an allergen derivative can be considered as hypoallergenic when the Z-score is significantly higher (i.e. at least 10%, preferably at least 20%, more preferably at least 50% higher) than the Z-score of the wild-type allergen. The Z-score threshold which is used to characterise an allergen derivative as hypoallergenic varies from allergen to allergen. Exemplarily, the following Z-score values define the threshold above which an allergen variant of a wild-type allergen can be considered as “hypoallergenic”.

Amb t 5 (hypoallergenic with a Z-score higher than −7.40, i.e. 10% higher than −6.66; 20% higher than −5.92)
Api g1 (hypoallergenic with a Z-score higher than −7.20)
Api m 1 (hypoallergenic with a Z-score higher than −4.60)
Api m 2 (hypoallergenic with a Z-score higher than −9.20)
Bet v 2 (hypoallergenic with a Z-score higher than −6.20)
Bet v 4 (hypoallergenic with a Z-score higher than −8.50)
Bla g 2 (hypoallergenic with a Z-score higher than −9.10)
Bos d 2 (hypoallergenic with a Z-score higher than −6.10)
Bos d 4 (hypoallergenic with a Z-score higher than −5.80)
Bos d 5 (hypoallergenic with a Z-score higher than −7.60)
Chi t 1 (hypoallergenic with a Z-score higher than −8.70)
Cyp c 1 (hypoallergenic with a Z-score higher than −7.50)
Der f 2 (hypoallergenic with a Z-score higher than −7.10)
Der f 13 (hypoallergenic with a Z-score higher than −6.10)
Der p 1 (hypoallergenic with a Z-score higher than −7.70)
Der p 2 (hypoallergenic with a Z-score higher than −5.80)
Equ c 1 (hypoallergenic with a Z-score higher than −7.10)
Fel d 1 (hypoallergenic with a Z-score higher than −5.80)
Gal d 2 (hypoallergenic with a Z-score higher than −10.80)
Gal d 3 (hypoallergenic with a Z-score higher than −15.00)
Gal d 4 (hypoallergenic with a Z-score higher than −8.00)
Glymlectin (hypoallergenic with a Z-score higher than −8.10)
Hev b 6 (hypoallergenic with a Z-score higher than −6.70)
Hev b 8 (hypoallergenic with a Z-score higher than −8.20)
Horv1 (hypoallergenic with a Z-score higher than −6.40)
Jun a 1 (hypoallergenic with a Z-score higher than −7.00)
Mus m 1 (hypoallergenic with a Z-score higher than −6.90)
Ole e 6 (hypoallergenic with a Z-score higher than −2.80)
Phl p 2 (hypoallergenic with a Z-score higher than −6.60)
Phl p 5 (hypoallergenic with a Z-score higher than −6.70)
Phl p 6 (hypoallergenic with a Z-score higher than −6.70)
Pru a v1 (hypoallergenic with a Z-score higher than −7.20)
Pru p 3 (hypoallergenic with a Z-score higher than −6.40)
Rat n 1 (hypoallergenic with a Z-score higher than −7.90)
Sola t 1 (hypoallergenic with a Z-score higher than −8.90)
Ves v 5 (hypoallergenic with a Z-score higher than −6.20)
Zea m14 (hypoallergenic with a Z-score higher than −7.50)

Another aspect of the present invention relates to a hypoallergenic derivative of a wild-type allergen exhibiting a three-dimensional structure, having a Z-score which differs from the three-dimensional structure of the wild-type allergen, and being derived from an allergen, is selected from the group consisting of Amb a 1, Amb a 2, Amb a 3, Amb a 5, Amb a 6, Amb a 7, Amb a 8, Amb a 9, Amb a 10, Amb t 5, Art v 1, Art v 2, Art v 3, Art v 4, Art v 5, Art v 6, Hel a 1, Hel a 2, Hel a 3, Mer a 1, Che a 1, Che a 2, Che a 3, Sal k 1, Cat r 1, Pla l 1, Hum j 1, Par j 1, Par j 2, Par j 3, Par o 1, Cyn d 1, Cyn d 7, Cyn d 12, Cyn d 15, Cyn d 22w, Cyn d 23, Cyn d 24, Dac g 1, Dac g 2, Dac g 3, Dac g 5, Fes p 4w, Hol l 1, Lol p 1, Lol p 2, Lol p 3, Lol p 5, Lol p 11, Pha a 1, Phl p 1, Phl p 2, Phl p 4, Phl p 5, Phl p 6, Phl p 11, Phl p 12, Phl p 13, Poa p 1, Poa p 5, Sor h 1, Pho d 2, Aln g 1, Bet v 1, Bet v 2, Bet v 3, Bet v 4, Bet v 6, Bet v 7, Car b 1, Cas s 1, Cas s 5, Cas s 8, Cor a 1, Cor a 2, Cor a 8, Cor a 9, Cor a 10, Cor a 11, Que a 1, Fra e 1, Lig v 1, Ole e 1, Ole e 2, Ole e 3, Ole e 4, Ole e 5, Ole e 6, Ole e 7, Ole e 8, Ole e 9, Ole e 10, Syr v 1, Cry j 1, Cry j 2, Cup a 1, Cup s 1, Cup s 3w, Jun a 1, Jun a 2, Jun a 3, Jun o 4, Jun s 1, Jun v 1, Pla a 1, Pla a 2, Pla a 3, Aca s 13, Blo t 1, Blo t 3, Blo t 4, Blo t 5, Blo t 6, Blo t 10, Blo t 11, Blo t 12, Blo t 13, Blo t 19, Der f 1, Der f 2, Der f 3, Der f 7, Der f 10, Der f 11, Der f 14, Der f 15, Der f 16, Der f 17, Der f 18w, Der m 1, Der p 1, Der p 2, Der p 3, Der p 4, Der p 5, Der p 6, Der p 7, Der p 8, Der p 9, Der p 10, Der p 11, Der p 14, Der p 20, Der p 21, Eur m 2, Eur m 14, Gly d 2, Lep d 1, Lep d 2, Lep d 5, Lep d 7, Lep d 10, Lep d 13, Tyr p 2, Tyr p 13, Bos d 2, Bos d 3, Bos d 4, Bos d 5, Bos d 6, Bos d 7, Bos d 8, Can f 1, Can f 2, Can f 3, Can f 4, Equ c 1, Equ c 2, Equ c 3, Equ c 4, Equ c 5, Fel d 1, Fel d 2, Fel d 3, Fel d 4, Fel d 5w, Fel d 6w, Fel d 7w, Cav p 1, Cav p 2, Mus m 1, Rat n 1, Alt a 1, Alt a 3, Alt a 4, Alt a 5, Alt a 6, Alt a 7, Alt a 8, Alt a 10, Alt a 12, Alt a 13, Cla h 2, Cla h 5, Cla h 6, Cla h 7, Cla h 8, Cla h 9, Cla h 10, Cla h 12, Asp fl 13, Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 15, Asp f 16, Asp f 17, Asp f 18, Asp f 22w, Asp f 23, Asp f 27, Asp f 28, Asp f 29, Asp n 14, Asp n 18, Asp n 25, Asp o 13, Asp o 21, Pen b 13, Pen b 26, Pen ch 13, Pen ch 18, Pen dh 20, Pen c 3, Pen c 13, Pen c 19, Pen c 22w, Pen c 24, Pen o 18, Fus c 1, Fus c 2, Tri r 2, Tri r 4, Tri t 1, Tri t 4, Cand a 1, Cand a 3, Cand b 2, Psi c 1, Psi c 2, Cop c 1, Cop c 2, Cop c 3, Cop c 5, Cop c 7, Rho m 1, Rho m 2, Mala f 2, Mala f 3, Mala f 4, Mala s 1, Mala s 5, Mala s 6, Mala s 7, Mala s 8, Mala s 9, Mala s 10, Mala s 11, Mala s 12, Mala s 13, Epi p 1, Aed a 1, Aed a 2, Api m 1, Api m 2, Api m 4, Api m 6, Api m 7, Bom p 1, Bom p 4, Bla g 1, Bla g 2, Bla g 4, Bla g 5, Bla g 6, Bla g 7, Bla g 8, Per a 1, Per a 3, Per a 6, Per a 7, Chi k 10, Chi t 1-9, Chi t 1.01, Chi t 1.02, Chi t 2.0101, Chi t 2.0102, Chi t 3, Chi t 4, Chi t 5, Chi t 6.01, Chi t 6.02, Chi t 7, Chi t 8, Chi t 9, Cte f 1, Cte f 2, Cte f 3, Tha p 1, Lep s 1, Dol m 1, Dol m 2, Dol m 5, Dol a 5, Pol a 1, Pol a 2, Pol a 5, Pol d 1, Pol d 4, Pol d 5, Pol e 1, Pol e 5, Pol f 5, Pol g 5, Pol m 5, Vesp c 1, Vesp c 5, Vesp m 1, Vesp m 5, Ves f 5, Ves g 5, Ves m 1, Ves m 2, Ves m 5, Ves p 5, Ves s 5, Ves vi 5, Ves v 1, Ves v 2, Ves v 5, Myr p 1, Myr p 2, Sol g 2, Sol g 4, Sol i 2, Sol i 3, Sol i 4, Sol s 2, Tria p 1, Gad c 1, Sal s 1, Bos d 4, Bos d 5, Bos d 6, Bos d 7, Bos d 8, Gal d 1, Gal d 2, Gal d 3, Gal d 4, Gal d 5, Met e 1, Pen a 1, Pen i 1, Pen m 1, Pen m 2, Tod p 1, Hel as 1, Hal m 1, Ran e 1, Ran e 2, Bra j 1, Bra n 1, Bra o 3, Bra r 1, Bra r 2, Hor v 15, Hor v 16, Hor v 17, Hon v 21, Sec c 20, Tri a 18, Tri a 19, Tri a 25, Tri a 26, Zea m 14, Zea m 25, Ory s 1, Api g 1, Api g 4, Api g 5, Dau c 1, Dau c 4, Cor a 1.04, Cor a 2, Cor a 8, Fra a 3, Fra a 4, Mal d 1, Mal d 2, Mal d 3, Mal d 4, Pyr c 1, Pyr c 4, Pyr c 5, Per a 1, Pru ar 1, Pru ar 3, Pru av 1, Pru av 2, Pru av 3, Pru av 4, Pru d 3, Pru du 4, Pru p 3, Pru p 4, Aspa o 1, Cro s 1, Cro s 2, Lac s 1, Vit v 1, Mus xp 1, Ana c 1, Ana c 2, Cit l 3, Cit s 1, Cit s 2, Cit s 3, Lit c 1, Sin a 1, Gly m 1, Gly m 2, Gly m 3, Gly m 4, Vig r 1, Ara h 1, Ara h 2, Ara h 3, Ara h 4, Ara h 5, Ara h 6, Ara h 7, Ara h 8, Len c 1, Len c 2, P is s 1, P is s 2, Act c 1, Act c 2, Cap a 1w, Cap a 2, Lyc e 1, Lyc e 2, Lyc e 3, Sola t 1, Sola t 2, Sola t 3, Sola t 4, Ber e 1, Ber e 2, Jug n 1, Jug n 2, Jug r 1, Jug r 2, Jug r 3, Ana o 1, Ana o 2, Ana o 3, Rlc c 1, Ses i 1, Ses i 2, Ses i 3, Ses i 4, Ses i 5, Ses i 6, Cuc m 1, Cuc m 2, Cuc m 3, Ziz m 1, Ani s 1, Ani s 2, Ani s 3, Ani s 4, Arg r, Asc s 1, Car p 1, Den n 1, Hev b 1, Hev b 2, Hev b 3, Hev b 4, Hev b 5, Hev b 6.01, Hev b 6.02, Hev b 6.03, Hev b 7.01, Hev b 7.02, Hev b 8, Hev b 9, Hev b 10, Hev b 11, Hev b 12, Hev b 13, Hom s 1, Rom s 2, Hom s 3, Hom s 4, Hom s 5 and Trip s 1 are preferably selected from the group consisting of Amb a 1, Art v 1, Par j 1, Cyn d 1, Dac g 1, Lol p 1, Phl p 1, Phl p 2, Phl p 4, Phl p 5, Phl p 6, Aln g 1, Bet v 1, Cas s 1, Cor a 1, Que a 1, Ole e 1, Cry j 1, Jun a 1, Der f 1, Der m 1, Der p 1, Equ c 1, Fel d 1, Alt a 1, Cla h 2, Asp f 1, Pen b 13, Cand a 1, Api m 1, Pol a 1, Vesp c 1, Bra j 1, Bra n 1, Bra o 3, Bra r 1, Zea m 14, Api g 1, Dau c 1, Mal d 1, Pru ar 1, Pru av 1, Ara h 1, Cap a 1w, Lyc e 1, Hev b 1, Hev b 2, Hev b 3, Hev b 4, Hev b 5, Hev b 6 and Hom s 1.

The Z-score of the hypoallergenic derivative is preferably increased for at least 5%, 6%, 7%, 8% or 10% compared to the Z-score of the wild-type allergen.

Surprisingly, it turned out that mutated molecules derived from said allergens and exhibiting a Z-score differing from the Z-score of wild-type allergens, were hypoallergenic.

According to a preferred embodiment of the present invention the allergen derivative comprises at least one mutation compared to the wild-type allergen.

It is preferred that the three-dimensional structure of the hypoallergenic molecule differs from that of the wild-type due to at least one mutation. The advantage of a low number of mutations (e.g. a maximum of 1, 2, 3, 4, 5, 7, 10, 15, 20, 30, 40, 50 mutations) is that the molecule itself and its epitopes, in particular its T-cell epitopes, remain substantially unaffected and consequently show T-cell responses which are comparable to those of a wild-type allergen, although the molecule itself is hypoallergenic. However, it is, of course, also possible to introduce a larger amount of mutations into the molecule, provided that said hypoallergenic molecule is still able to induce an allergen-specific response when administered to an individual.

According to a further preferred embodiment the at least one mutation is selected from the group consisting of amino acid exchange, amino acid deletion and amino acid insertion.

The type of mutation introduced into the molecule may be of any kind, in particular amino acid exchange, amino acid deletion and amino acid insertion are preferred.

The hypoallergenic molecule of the present invention is preferably derived from the allergen Bet v 1.

According to another preferred embodiment of the present invention the derivative is selected from the group consisting of SEQ ID No. 2, SEQ ID No. 3 and SEQ ID No. 4.

In particular, these Bet v 1 derivatives induce cellular immune response when administered to a patient, and do not bind IgE as efficient as wild-type Bet v 1. Therefore, these hypoallergenic molecules are particularly preferred.

Further aspects of the present invention relate to a vaccine formulation comprising a hypoallergenic derivative according to the present invention and to the use of a hypoallergenic derivative according to the present invention for the manufacture of a vaccine for preventing and treating allergies.

The hypoallergenic derivatives of the present invention can be used for manufacturing vaccine formulations. It is well known in the art that hypoallergenic molecules derived from an allergen can be used for the prevention, treatment or desensibilisation of individuals who suffer from or are susceptible to an allergic condition. The route of administration of such vaccine formulations may include injection via the intramuscular, intraperitoneal, intradermal or subcutaneous routes, or via mucosal administration to the oral/alimentary, respiratory, genitourinary tracts. A preferred route of administration is via the transdermal route, for example by skin patches.

In order to enhance the immune response against the hypoallergenic molecule of the present invention, said molecule may be administered together with an adjuvant. Said adjuvant may be conjugated to the hypoallergenic molecule or bound thereto covalently (e.g. recombinantly).

Vaccine preparation is generally described in “New Trends and Developments in Vaccines”, ed by Voller et al., University. Park Press, Baltimore, Md., USA 1978. Conjugation of proteins to macromolecules is disclosed by Likhite, U.S. Pat. No. 4,372,945 and by Armor et al., U.S. Pat. No. 4,474,757.

The present paper describes an approach which enables the routine in silico creation and evaluation of hypoallergenic allergen derivatives. The computational procedure uses knowledgebase potentials (KBPs). KBPs have a broad range of applications in computational structural biology. In general, they can be used to investigate sequence structure relations in known 3D protein structures. The potential effectively describes the interactions between amino acid residues (pair term) and the interaction of the residues with the surrounding solvent (surface term). Recently, KBPs have been shown to be a valuable tool for in silico mutation experiments, where they can be used to estimate structural changes of the protein structures under sequence variation. Calculation and comparison of energy differences between wild-type and mutant sequence, expressed in Z-scores, predict the influence of the mutation on the protein stability. An increased Z-score indicates destabilisation and thus a potential change in the 3D structure.

We selected two mutants of the birch pollen allergen Bet v 1a fulfilling the necessary criterion, i.e. a lowered Z-score indicating structural changes, and tested these molecules with respect to their immunogenicity and allergenicity. The predicted hypoallergenicity was confirmed by the experimental evaluation.

The present invention is further illustrated by the following figures and examples, however, without being restricted thereto.

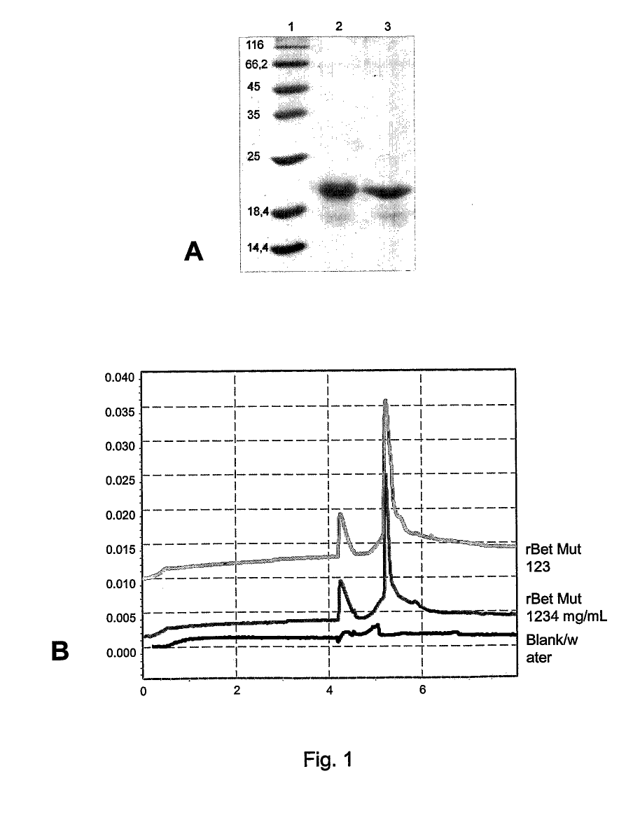

FIG. 1 shows an SDS-PAGE and CE of the mutated proteins. Protein purity was determined on a 15% SDS-PAGE gel stained with Coomassie. Lane 1: molecular weight protein marker (sizes given in kD), lane 2: rBet Mut 123, lane 3: rBet Mut 1234 (A). CE elution profile of the two mutant proteins. x-axis=elution time (minutes), y-axis=UV-absorption (210 nm) (B).

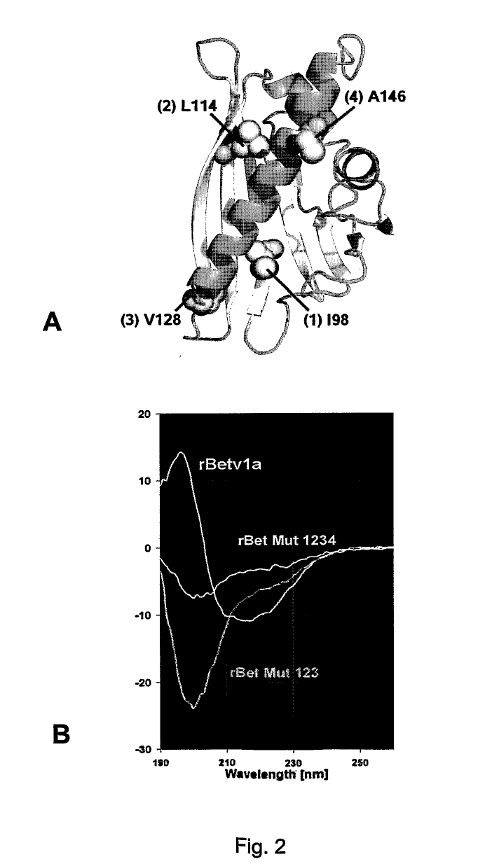

FIG. 2 shows structure and CD spectra analysis. Cartoon representation of the wild-type Betv (1BV1). The four mutation sites are shown with spheres (A). The different courses of the spectra analysis of the mutants rBet Mut 123 and rBet Mut 1234 indicate a structural change that occurred as a consequence of the mutations (B).

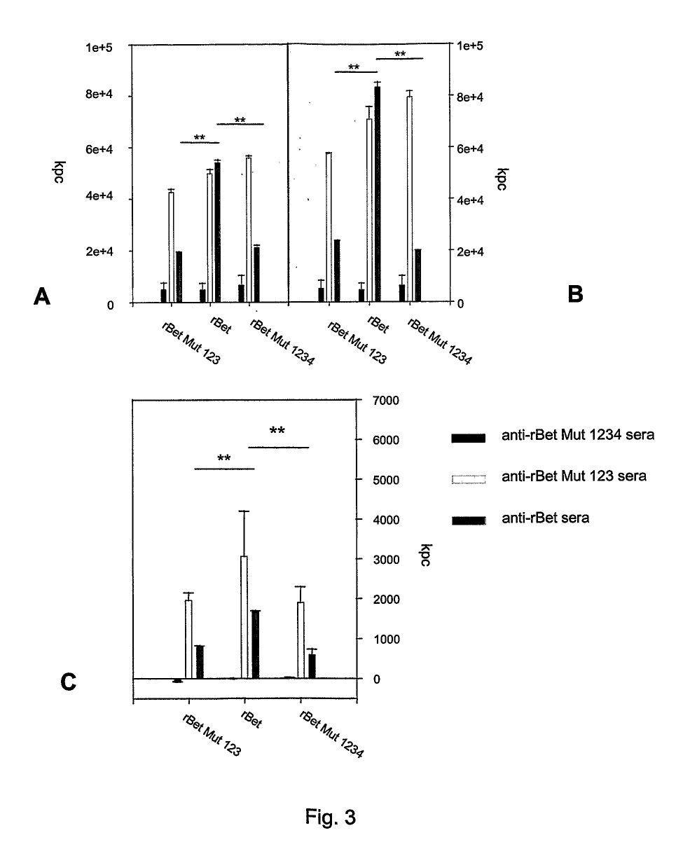

FIG. 3 shows antibody responses against wild type and mutant proteins. Specific antibodies directed against rBet v 1a, rBet Mut 123 and rBet Mut 1234 were determined by ELISA (total IgG (A), IgG1 (B) and IgG2a (C)). Mice were immunised twice in a weekly interval and sera were taken two weeks after the second protein immunisation. Data are shown as Δ-pre-serum values in kilophoton counts/s (kpc) on the y-axis and expressed as mean±SEM (**p<0.0001) 4 shows an RBL cell release and release-inhibition assay. Anaphylactic sera specific for rBet v 1a were obtained by immunising mice, twice with 5 μg rBet v 1a in a weekly interval. RBL cells were passively sensitised with pooled sera of these mice and crosslinking was performed with either rBet v 1a, rBet Mut 123 or rBet Mut 1234 (A). Sera from mice immunised twice with rBet, rBet Mut 123 or rBet Mut 1234 served for passive sensitisation of RBL cells and crosslinking with the wild-type rBet v 1a (B). The same sera and rBet v 1a were used to assess the presence of IgE-epitope blocking antibodies in an inhibition RBL assay (C). Data are shown as percent β-hexosaminidase release on the y-axis, data are expressed as mean±SEM. ** p<0.01, * p<0.05)

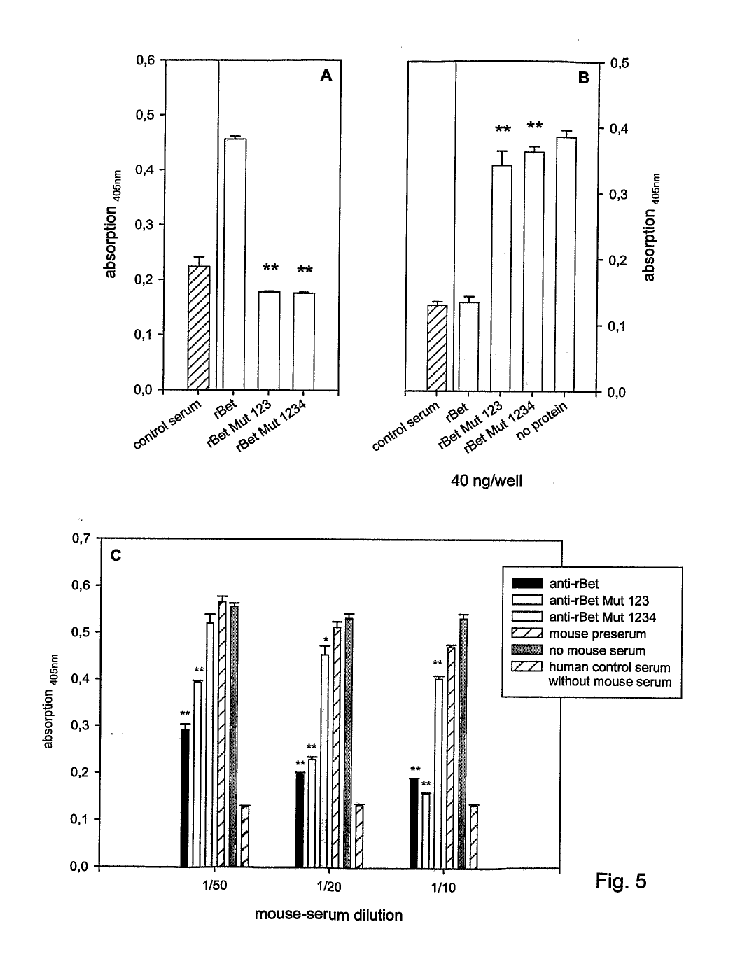

FIG. 5 shows the binding and inhibition assays with IgE antibodies from a birch pollen allergic patient's serum pool. Binding of IgE antibodies from a pool of 42 birch pollen allergic patients to rBet v 1a or the mutants rBet Mut 123 or rBet Mut 1234 was assessed with an ELISA (A). A competition ELISA was performed to ensure that the reduced IgE binding of the mutants observed was not due to varying plate-binding properties of the different recombinant proteins (B). IgE epitope blocking antibodies were detected in an inhibition ELISA experiment (C). Data are expressed as absorption at 405 nm and mean±SEM. ** p<0.01 * p<0.05. Significances are related to rBet (A and B) and to mouse preserum (C).

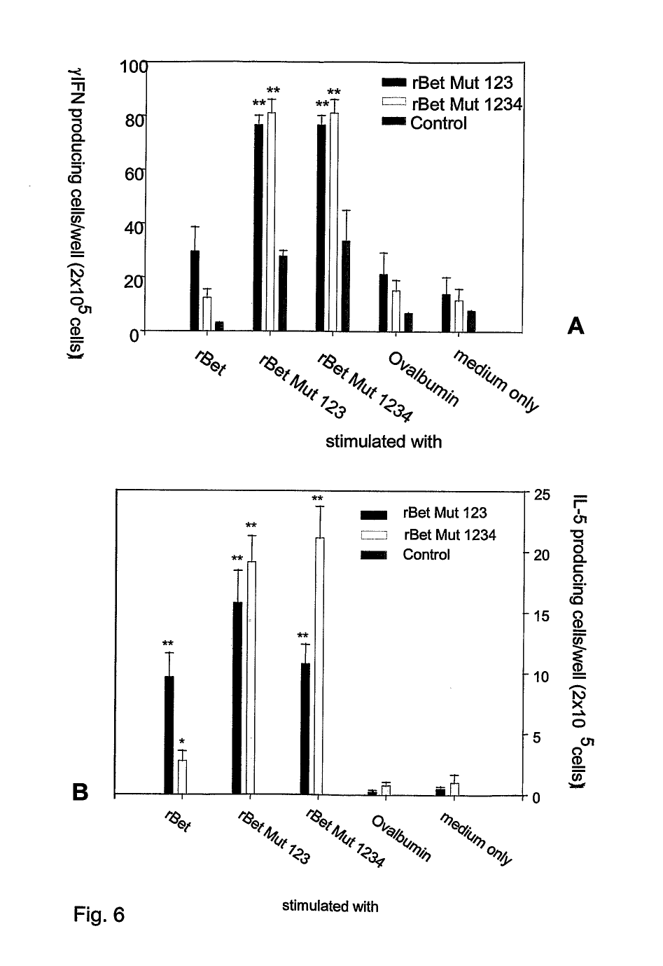

FIG. 6 shows IFN-7 and IL-5-producing cells in spleens of immunised mice. An ELISPOT assay was carried out to detect IFN-γ (A) and IL-5 (B) producing cells. Spleen cells of treated mice were harvested on day 28, sown into membrane-bottomed 96 well plates and cultured in the presence of antigen for 24 hours. The assay was performed as described and the spots were counted. Data are expressed as mean±SEM. ** p<0.01 * p<0.05. Significances are related to ovalbumin stimulated cells.

### EXAMPLES

The concept of component-resolved diagnosis and therapy of allergic diseases is based on the production of purified allergens. Based on natural hypoallergenic isoforms, a full-length Bet v 1 hypoallergen was engineered by amino acid substitution. Site-directed mutagenesis was used for a number of allergens to create hypoallergenic derivatives, such as mutated Ara h 1-3, Mal d 1, Lol p 5 and Hev b 5. Another type of approach to make allergens hypoallergenic was to fragment, fuse or shuffle entire molecules or parts of them, e.g. fragmentation of Bet v 1a and Der f 2 fusion of copies of Bet v 1, hybrid and chimeric molecules.

In the present invention and in the following examples a novel approach to the routine screening and production of hypoallergenic derivatives of recombinant allergens applicable for SIT is described. The computational procedure using knowledge based potentials enables in silico mutation and screening of allergens with structural changes, the destabilisation degree of which is indicated by the Z-scores. The aim of the example was to locate single sites in the protein structure where a mutation has the maximum effect. For this purpose, an experimentally determined 3D structure per se is very useful to guess reasonable mutation sites, because it provides information about proximities in space and the like if a residue is in the core or exposed on the surface. In the present example also the interaction energies and the effect of single point mutations on protein stability were analysed. This led to the identification of four point mutations which decrease most of the interaction energies, here approximately by 0.5 Z-score units, either in the combined energies or in a single interaction type. The most destabilising effects are caused by mutations in the protein core. With the in silico approach it has been possible to find out which core residues are most sensitive for destabilising or stabilising mutations and which replacement amino acid has the strongest effect.

The experimental data confirmed that destabilisation of the native protein structure should lead to the loss of epitopes, thus resulting in hypoallergenic derivatives with reduced capacity to crosslink pre-existing IgE on mast cells and basophils.

The Z-scores of the mutated proteins correlated with their hypoallergenic and immunogenic nature. Already a single mutation increased the Z-scores, wherein three or four mutations showed a marked increase of the Z-scores. The immunogenicity of the derivative containing four mutations was drastically reduced with respect to antibody induction (FIG. 3), indicating a massive destabilisation of the protein folding. Nevertheless, the cellular immunogenicity of this mutant concerning T cell activation and cytokine production was maintained (FIG. 6). Moreover, antibodies directed against the wild type allergen recognised the mutant (FIG. 3) indicating a minimal maintenance of native epitopes.

The hypoallergenicity of the mutants was proven by RBL cell release and release inhibition assays with mouse sera, and ELISA binding, inhibition and competition assays with a human serum pool. Both mutants, expressed as recombinant proteins, clearly revealed a reduced capacity to crosslink IgE antibodies directed against the wild type allergen in the mouse model. Furthermore, the ELISA experiments with a pool of sera from allergic patients showed a reduced IgE-binding of the mutants, thus indicating the increased safety profile of the mutants for clinical use. In addition to the hypoallergenic nature and T cell reactivity, the derivate containing three mutations induced antibodies with a significant blocking activity as measured with a RBL release-inhibition assay.

Bet Mut 1234 induced no IgG1 and IgG2a antibody responses (FIG. 3) and also completely lacked IgE production as measured by the highly sensitive RBL cell release assay. Thus, both, crosslinking of pre-existing IgE as well as new synthesis of IgE can be excluded with this molecule.

The method of the present invention using knowledge-based potentials for mutation and pre-screening of molecules opens a wide application for creating hypoallergenic, safety-optimised vaccine candidates for SIT. The only restriction of this method, i.e. knowledge of the three-dimensional structure of the molecule, seems to be no major problem because the number of allergen structures available in the data banks is already high and growing with increasing speed.

Based on a clear rationale, the approach has several advantages over the hitherto applied methods for the development of recombinant hypoallergens. In general, it enables to select suitable mutant molecules by clearly defined parameters, the Zscores. In contrast thereto, the success of the above mentioned attempts to create hypoallergens, e.g. site directed mutagenesis or the insertion of point mutations learned from natural hypoallergens is more or less pure coincidence. The conditions which lead to the hypoallergenicity of these molecules are neither known nor predictable which means that numerous time-consuming and blind attempts must be made to finally find suitable candidate molecules. The situation is similar with approaches using fragments, fusion or hybrid molecules. None of these approaches is knowledge-based but mostly, fragmentation sites and fusion partner molecules are selected by arbitrary decisions. The general problems related with fragmentation and fusion can be illustrated by the following facts: Fragmentation of Bet v 1 clearly resulted in two hypoallergenic fragments which display a different structure than the entire wild type molecule. However, various fragmentations of Phl p 5 do not yield any hypoallergenic derivative at all, thus indicating that the fragmentation approach cannot be generalised. Moreover, a loss of allergenicity by fragmentation depends on the molecular context, e.g. within a hybrid fusion molecule of Phl p 5 and Bet v 1 fragments, the originally hypoallergenic Bet v 1 fragments regained their allergenicity, obviously by refolding within this specific molecular context.

A major benefit of our knowledge-based in silico approach is that the mutation and selection criteria can be easily adopted and/or updated with any knowledge and new data about structural features and/or relevant T- and/or B cell epitopes of allergens, thus ensuring the production of state-of-the-art hypoallergens for clinical use.

Summing up, the present data demonstrate that destabilising the structure of allergens using in silico mutation and screening offers a reliable routine method to pre-select panels of hypoallergenic vaccine candidates with defined molecular properties.

### Example 1

### Construction of Expression Vectors

For the pHIS-Bet Mut 1234 vector a PCR reaction using pCMV-Bet as a template with the forward primer 5′-CACCGAATTCATGGGTGTTTTCAATTACGA-3′ (SEQ ID No. 5) and the reverse primer 5′-CGACTCTAGACATGGTCACCTTTGGTGTGGTACTTGTTGCTGATCTTCTTGATGGATCCTCCATCAGGGGTTGCCACTATCTTTATCTCGTTGGACTTCTTCTCCAATG-3′ (SEQ ID No. 6) was performed, yielding a 392 by (base pair) fragment. This fragment was the template for another PCR reaction using the same forward primer as before and the reverse primer 5′-CGACTCTAGATTAGTTGTAGGCATCGGAGTGTGCCAAGAGGTAGCTCTCAACTGGCCTCAAAAGTGTCTCGCCCATTTCTTTACTTGCCTTAACCTGCTCTGCCTTCTCCTCATGGTCACCTTTGGTG-3′ (SEQ ID No. 7). The resulting fragment was Eco RI-Xba I digested and cloned into a pCi Genbank vector (Promega, USA) which was digested with the same enzymes. The last step was the PCR amplification of the coding region of this vector using the forward primer 5′-CACCATGGGTGTTTTCAATTACGA-3′ (SEQ ID No. 8) and the reverse primer 5′-GATCGAATTCTTAGTTGTAGGCATCGGAGT-3′ (SEQ ID No. 9). The resulting fragment was subcloned into a NcoI-EcoRI digested pHIS parallel II vector.

Generating the pHIS-Bet Mut 123 vector was a four-step process. For the first step pCMV-Bet was used as a template for two separate PCR reactions, one using the forward primer 5′-ATTGGAGAAGAAGTCCAACGAGAT-3′ (SEQ ID No. 10) and the reverse primer 5′-GATCTCTAGATTAGTTGTAGGCATCGGAGTG-3′ (SEQ ID No. 11) and the other one using the forward primer 5′-CACCGAATTCATGGGTGTTTTCAATTACGA-3′ (SEQ ID No. 12) and the reverse primer 5′-ATCTCGTTGGACTTCTTCTCCAAT-3′ (SEQ ID No. 13). A third PCR reaction was performed with the forward primer 5′-CACCGAATTCATGGGTGTTTTCAATTACGA-3′ (SEQ ID No. 12) and the reverse primer 5′-GATCTCTAGATTAGTTGTAGGCATCGGAGTG-3′ (SEQ ID No. 14). The two PCR fragments were used as templates. The resulting PCR fragment was subcloned into an EcoRI-XbaI-digested pCi vector.

The resulting vector was used as a template for two separate PCR reactions, one using the forward primer 5′-AGGATCCATCAAGAAGATCAGC-3′ (SEQ ID No. 15) and the reverse primer 5′-GATCTCTAGATTAGTTGTAGGCATCGGAGTG-3′ (SEQ ID No. 16) and the other one using the forward primer 5′-CACCGAATTCATGGGTGTTTTCAATTACGA-3′ (SEQ ID No. 12) and the reverse primer 5′-GCTGATCTTCTTGATGGATCCT-3′ (SEQ ID No. 17). A third PCR reaction was performed with the forward primer 5′-CACCGAATTCATGGGTGTTTTCAATTACGA-3′ (SEQ ID No. 12) and the reverse primer 5′-GATCTCTAGATTAGTTGTAGGCATCGGAGTG-3′ (SEQ ID No. 18) with the two PCR fragments as templates. The resulting PCR fragment was subcloned into an EcoRI-XbaI-digested pCi vector.

This construct was used as a template for two separate PCR reactions, one using the forward primer 5′-TGACCATGAGGAGAAGGCAGAG-3′ (SEQ ID No. 19) and the reverse primer 5′-GATCTCTAGATTAGTTGTAGGCATCGGAGTG-3′ (SEQ ID No. 16) and the other one using the forward primer 5′-CACCGAATTCATGGGTGTTTTCAATTACGA-3′ (SEQ ID No. 12) and the reverse primer 5′-CTCTGCCTTCTCCTCATGGTCA-3′ (SEQ ID No. 20). A third PCR reaction was performed with those two fragments and the forward primer 5′-CACCGAATTCATGGGTGTTTTCAATTACGA-3′ (SEQ ID No. 12) and the reverse primer 5′-GATCTCTAGATTAGTTGTAGGCATCGGAGTG-3′ (SEQ ID No. 18). The resulting PCR fragment was subcloned into an EcoRI-XbaI-digested pCi vector.

The final step was the PCR amplification of the coding region of this vector, conducted with the forward primer 5′-CACCATGGGTGTTTTCAATTACGA-3′ (SEQ ID No. 12) and the reverse primer 5′-GATCGAATTCTTAGTTGTAGGCATCGGAGT-3′ (SEQ ID No. 21). The resulting fragment was subcloned into a NcoI-EcoRI digested pHIS parallel II vector.

Protein sequences of the resulting proteins are shown in Table 1. The following amino acids were mutated at the following positions: 198K (1), L98K (2), L114K (3) and A146P (4).

TABLE 1

Protein sequences of the wild type rBet v1a and the mutant proteins:
WT rBet v1a (SEQ ID No. 1), rBet Mut 123 (SEQ ID No. 2), rBet Mut 1234
(SEQ ID No. 3), rBet Mut 4 (SEQ ID No. 3)

| | |
|---|---|
| Molecule | Sequence alignment |

| | |
|---|---|
| WT rBet v1a | GVFNYETETTSVIPAARLFKAFILDGDNLFPKVAPQAISSVENIEGNGGPGTIKKISFP |
| rBet Mut 123 | GVFNYETETTSVIPAARLFKAFILDGDNLFPKVAPQAISSVENIEGNGGPGTIKKISFP |
| rBet Mut 1234 | GVFNYETETTSVIPAARLFKAFILDGDNLFPKVAPQAISSVENIEGNGGPGTIKKISFP |
| rBet Mut 4 | GVFNYETETTSVIPAARLFKAFILDGDNLFPKVAPQAISSVENIEGNGGPGTIKKISFP |
| WT rBet v1a | EGFPFKYVKDRVDEVDHTNFKYNYSVIEGGPIGDTLEK I SNEIKIVATPDGGSI L KISN |
| rBet Mut 123 | EGFPFKYVKDRVDEVDHTNFKYNYSVIEGGPIGDTLEK K SNEIKIVATPDGGSI K KISN |
| rBet Mut 1234 | EGFPFKYVKDRVDEVDHTNFKYNYSVIEGGPIGDTLEK K SNEIKIVATPDGGSI K KISN |
| rBet Mut 4 | EGFPFKYVKDRVDEVDHTNFKYNYSVIEGGPIGDTLEK I SNEIKIVATPDGGSI L KISN |
| WT rBet v1a | KYHTKGDHE V KAEQVKASKEMGETLLR A VESYLLAHSDAYN |
| rBet Mut 123 | KYHTKGDHE E KAEQVKASKEMGETLLR A VESYLLAHSDAYN |
| rBet Mut 1234 | KYHTKGDHE E KAEQVKASKEMGETLLR P VESYLLAHSDAYN |
| rBet Mut 4 | KYHTKGDHE V KAEQVKASKEMGETLLR P VESYLLAIISDAYN |

### Example 2

### Immunisation Experiments

Female BALB/c mice (6-8 weeks of age) were immunised with the wild-type rBet via or the recombinant mutant Bet proteins rBet Mut 123 and rBet Mut 1234. Groups of 4 female BALB/c mice each were immunised twice in a weekly interval via subcutaneous (s.c.) injection of 5 μg purified protein in sterile PBS with 100 μl Al(OH)₃ (Serva, Germany) in a total volume of 150 μl into two spots on the back. The mice were sacrificed 28 days after the first protein immunisation.

### Example 3

### Serology

IgG, IgG1 and IgG2a serum antibody levels were determined by a luminescence-based ELISA as described in Hartl A et al. (Methods 2004; 32:328-39).

### Example 4

### Lymphocyte Cultures

Culture of splenocytes was performed as described in Hochreiter R et al. (Eur J Immunol 2003; 33:1667-76) (except that 1% mouse serum instead of 5% calf serum was used and cells were plated at a density of 2×10⁵ cells per well). Cells were stimulated with recombinant antigen at a concentration of 20 μg/ml for 72 h.

### Example 5

### ELISPOT Assay

Lymphocytes prepared as above were cultured in anti-IFN-γ or IL-5 (clones AN-18.17.24 and TRFK5, 4 μg/ml) coated ELISPOT plates (Millipore, Austria) with 20 μg/ml antigen for 24 h as described for proliferation cultures. Cytokines were detected with biotinylated mAbs (2 μg/ml, clones R4-6A2 and TRFK4) followed by streptavidin-HRP (1:1000, Becton Dickinson Pharmingen, Austria,). The assay was developed using 3-amino-9-ethyl-carbazole substrate (Acros, Belgium).

### Example 6

### β-Hexosaminidase Release from Rat Basophil Leukemia Cells (RBL Assay)

As a functional read-out for IgE-mediated degranulation, a β-hexosaminidase release assay was performed using RBL-2H3 cells as described in Hochreiter R et al. (Eur J Immunol 2003; 33:1667-76). For the inhibition assays the antigen (3 ng/ml) used for the crosslinking was pre-incubated with 0, 2, 5 or 10% serum (inactivated at 56° C. for 1.5 hours) for 2 hours.

### Example 7

### ELISA with Human Sera

The serum used was pooled from 42 birch pollen allergic patients with RAST>4. The serum of a non-allergic patient served as a control. 96-well high-bind immunoplates (NUNC, Denmark) were coated by overnight incubation at 4° C. with 200 ng antigen/well in PBS and ELISA was performed as described in Hartl A et al. (Methods 2004; 32:328-39). As detection antibody alkaline phosphatase-conjugated anti-human IgE antibody (SigmaAldrich, Austria) was used. The assay was developed with AP-substrate (10 mM 4-nitrophenylphosphate disodium salt hexahydrate dissolved in 0.1M diethanolamine, 5 mM MgCl₂) and absorption measured at 405 nm.

For the competition assay sera were pre-incubated with 40 ng/well protein. The inhibition assay was performed by incubating plates with mouse sera prior to the addition of the human serum pool.

### Example 8

### Protein Purification of HIS-taq Proteins rBet Mut 1234 and rBet Mut 123

E. coli (BL21 DE3) were transformed with pHIS-Bet Mut 123 or 1234. A single clone was picked and cultured in LB/amp medium (100 μg/ml ampicillin) over night (37° C.). The culture was diluted 1:10 in LB amp medium and cultured until an OD₆₀₀ nm of 0.8 was reached. The induction was performed by addition of 0.1 mM IPTG (isopropyl-beta-D-thiogalactopyranosid). Bacteria were cultured for three hours at 37° C. and harvested by centrifugation. The pellet was resuspended in 1/50 volume lysis buffer (300 mM NaCl, 25 mM NaH₂PO₄, 10 mM imidazole)+10% glycerol and 1 mg/ml lysozyme. After 4-6 freeze-and-thaw cycles DNA was digested with 10 μg/ml DNAse I+1 mM MgCl₂, the suspension centrifuged and the pellet resuspended in 8 M Urea. DNA was cut up by ultrasound treatment on ice. After centrifugation, the supernatant was shock-lysed with lysis buffer, centrifuged again and the supernatant was loaded on the Ni-CAM™ HC Resin-column (Sigma, Germany). After a wash with lysis buffer, protein was eluted in elution buffer (300 mM NaCl, 25 mM NaH₂PO₄, 250 mM imidazole). The 6× histidin-tag was removed by digestion with 0.25 mg TEV (Tobacco Etch Virus) protease per 0.1 mg protein over night at room temperature. After dialyses against lysis buffer, the 6α-histidin-tag and the enzyme were removed from the same column. Finally, the protein solution was dialysed against distilled water. Protein concentration was then analysed by spectrophotometry at OD 280 nm and purity was assessed by SDS PAGE.

### Example 9

### Capillary Electrophoresis (CE)

The CE measurements were performed with the separation P/ACE 5000 system of Beckmann (USA), equipped with a photodiode array (PDA) detector. The data wer analysed with a P/ACE Station™ Version 1.2. Capillary dimensions were 50 μm i.d. and 375 μm o.d. with an effective length of either 40 or 50 cm and a total length of 47 or 57 cm.

The samples were injected hydrodynamically at 0.5 psi for 5-10 seconds and the separations were run at 15 or 20 kV at 20-35° C. The UV absorption was measured at 210 nm.

### Example 10

### Circular Dichroism (CD)

The CD spectra were measured with a Jasco J-810 spectropolarimeter and treated by means of the Spectra Manager, Version 1.53.00 (both Jasco, Japan).

Before starting the measurement, the system is flushed with nitrogen for 5 minutes. CD spectra are recorded in the far-UV region between 190-260 nm with a data pitch of 1 nm employing cuvettes with 1 mm path length. The secondary structures were calculated from CD spectra by means of one or all of the following programmes (SELCON3, CONTINLL, CDSSTR).

### Example 11

### Destabilising Mutations of Bet v 1

The strategy for finding destabilising mutations was to calculate all possible single site mutations and to combine them to a multi-site mutation. The computational effort to calculate multi-site mutations concurrently increases exponentially with the number of sites and is thus not feasible. In the worst case, our strategy would predict mutations which complement each other, such that the protein does not lose stability. This is, however, very unlikely. In fact, the Z-score increase of the Bet Mut 1234 is four times larger than for any single mutation. The Z-scores of the mutated proteins rBet Mut 123 and rBet Mut 1234 (Table 2) show an increase of about two units, which points to a significant decrease in the stability or to a change of the native structure of the proteins.

TABLE 2
Summary of the calculated Z-scores for the native
rBet v 1a and the mutant proteins rBet Mut 123,
rBet Mut 1234 and rBet Mut 4.

| | Combined Z-score | Pair Z-score | Surface Z-score |
|---|---|---|---|
| rBet v 1a | −9.18 | −6.45 | −7.01 |
| rBet Mut 123 | −7.31 | −5.64 | −5.50 |
| rBet Mut 1234 | −7.08 | −5.64 | −5.52 |
| rBet Mut 4 | −8.98 | −5.98 | −7.06 |

### Example 12

### Expression of rBet Mut 123 and rBet Mut 1234

The mutated proteins were expressed in the E. coli strain BL21(DE3) together with a 6× histidin-tag and purified from the inclusion bodies because of the higher yield and quality of the so, won proteins. Subsequently, the histidin-tag was removed by digestion with TEV protease and the solution was dialysed against distilled water. SDS-PAGE and CE (FIG. 1) demonstrate that both recombinant proteins could be isolated with high purity.

### Example 13

### Structural Analysis of rBet Mut 123 and rBet Mut 1234

The CD data showed that the Bet v 1a mutants folded differently than wild-type rBet v 1a. The content of α-helices is significantly decreased, while the β-sheet percentage slightly increases. This points to a changed structure of the mutated proteins. (FIG. 2, summarised in Table 3).

TABLE 3
Summary of the percentages of structural features
of the proteins obtained by CD analysis.

| Structural feature | rBet v 1a | rBet Mut123 | rBet Mut 1234 |
|---|---|---|---|
| α-helix | 14% | 5.5% | 4.6% |
| β-sheets | 35.7% | 38.7% | 41.2% |
| turns | 20.8% | 21.9% | 21.7% |
| random coil | 28.4% | 34% | 32.5 |

### Example 14

### The Mutated Proteins Display Altered Antigenicity and Immunogenicity

To evaluate the potential of the mutated proteins to be recognised by antibodies raised against the wild-type protein, an ELISA using polyclonal antibodies against the wild-type Bet v 1a was performed (FIG. 3.). The data showed that binding of rBet Mut 123 and rBet Mut 1234 by anti-rBet v 1a-specific total IgG antibodies was drastically reduced in comparison with the binding of the homologous wild-type rBet. This reduction was observed with total IgG but also with the antibody subclasses IgG1 and IgG2a. Furthermore, the immunogenicity of the mutants was compared to that of the wild-type allergen by two injections with 5 μg purified protein together with 100 μl of Al(OH)₃ (FIG. 3). Immunisation with the Bet v 1a protein containing three mutations (rBet Mut 123) induced antibodies which equally recognised the homologous molecule used for the immunisation as well as the wild-type protein and the protein containing an additional mutation (rBet Mut 1234). Again, this effect was observed with a similar pattern for the total IgG and the subclasses IgG1 and IgG2a (FIG. 3A-C).

Immunisation with rBet Mut 1234, however, triggered only a marginal humoral immune response, both, against the homologous protein as well as the second mutant protein or the wild-type allergen.

### Example 15

### Bet Mut 123 has a Reduced IgE Crosslinking Capacity and does not Trigger New IgE Production but Induces Blocking Antibodies

Crosslinking of pre-existing IgE with allergens is the major cause of anaphylactic reactions in allergic individuals undergoing SIT. Hypoallergenicity is therefore considered as a necessary prerequisite for future therapeutic approaches with recombinant allergens. Hypoallergenicity of the mutant proteins with respect to binding to and crosslinking of IgE antibodies directed against the native wild-type allergen was tested with RBL assays.

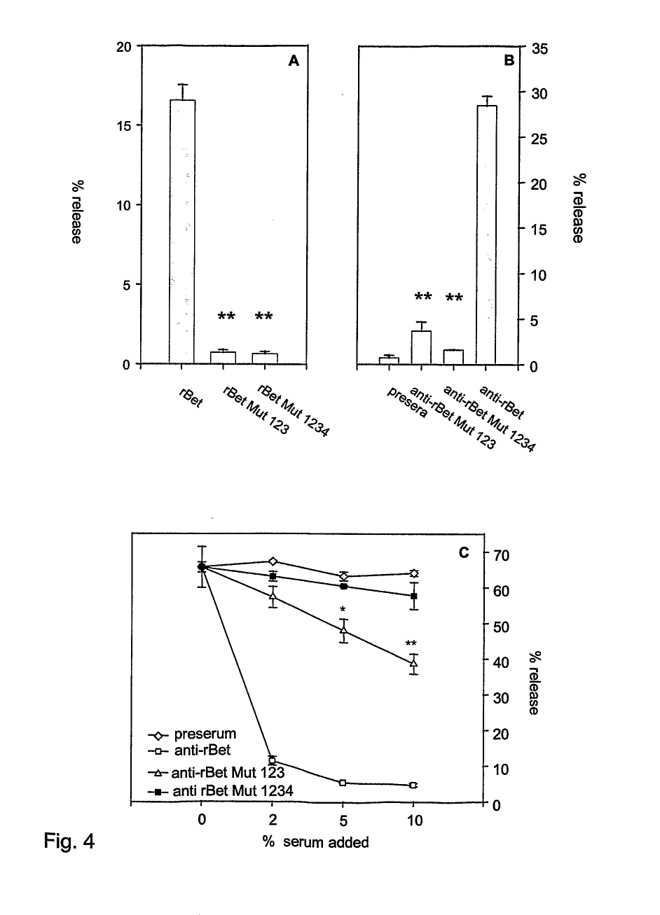

For this purpose, the RBL cells were passively sensitised with sera from mice immunised with the wild-type protein. The crosslinking of bound IgE was performed with either rBet v 1a or the two mutant proteins. The data revealed that rBet Mut 123 and rBet Mut 1234 show a significantly (for both p<0.01) reduced capability to cross-link rBet v 1a-specific IgE compared with the wild-type protein (FIG. 4a).

Further, the question was addressed, whether immunisation with the recombinant mutant proteins triggers IgE antibodies and if these were able to be cross-linked with the native allergen. The RBL assay performed with sera of these mice demonstrates that only negligible levels of IgE antibodies are triggered which can be cross-linked by the wild-type rBet v 1a (FIG. 4b).

In a third set of experiments the antibodies against the native allergen and the mutants were tested for their capacity to inhibit RBL cell release by blocking antibodies. RBL cells were coated with IgE against the native rBet v 1a and crosslinking was triggered with a solution containing the native rBet v 1a and different sera. The results showed a complete inhibition of the release with the homologous antiserum (anti-rBet) and a significant effect (p<0.05 at 5% and p<0.01 at 10% serum added) with the anti-rBet Mut 123 sera indicating the induction of blocking antibodies (FIG. 4c).

### Example 16

### Both Mutants of rBet v 1a are Hypoallergenic Concerning their Capacity to Bind Human IgE Antibodies

In addition to the hypoallergenicity proven with Bet v 1a-specific mouse IgE antibodies, both mutants were tested for their capacity to bind human IgE antibodies. A pool of sera from 42 exclusively birch pollen allergic patients with RAST>4 was used in ELISA plates coated with rBet v 1a and the mutants. Both derivatives showed a significantly reduced (p<0.01) binding of human IgE antibodies compared to that of the wild-type allergen (FIG. 5a). In order to rule out a different plate-binding behaviour of the mutant proteins as the cause for this differences, a competition ELISA was performed (FIG. 5b). For this purpose, the human sera were pre-incubated with the wild-type protein and the derivatives before adding to the microtiter plates which were coated with rBet v 1a. The data demonstrate that the mutant proteins were not able to compete with rBet v 1a for the binding of human IgE antibodies.

Similar to the RBL inhibition approach with mouse sera, the presence of Bet v 1a-specific IgE-epitope-blocking mouse antibodies induced with the wild-type allergen or the mutant proteins was detected by ELISA. Microtiter plates were coated with rBet v 1a and incubated with the different mouse antisera. The human serum pool was added in a third step and the blocking effect of the mouse sera was measured (FIG. 5c). The results demonstrate that the mouse serum won by immunisation with rBet v 1a as well as that won with the derivative containing three mutations (rBet Mut 123) reduced the binding of the human IgE antibodies. The blocking effect of the mouse serum directed against the derivative containing four mutations (rBet Mut 1234) was much less pronounced but still significant.

By this way rBet v 1a-specific antibodies in the mouse serum bind to rBet v 1a and block binding of human rBet v 1a-specific IgE. The data clearly show a significant reduction (p<0.01 for all dilutions) of the absorption/signal if the wells are pre-incubated with serum won from rBet Mut 123-immunised mice and a slight but statistically significant reduction with serum of rBet Mut 1234-immunised mice (FIG. 5c).

### Example 17

### The Mutated Allergens Induce Cellular Immune Responses

The humoral immunogenicity of the wild type allergen and the mutated derivatives has been investigated and shown in FIG. 3. The serological analysis showed that the mutant proteins were immunogenic if applied with adjuvants (Al(OH)₃), indirectly indicating the activation of T helper cells. To further assess the T cell immunogenicity of these proteins, an ELISPOT assay detecting secreted IFN-γ and IL-5, two key cytokines for Th1 or Th2, was performed. For this purpose, spleen cells of immunised animals were harvested, re-stimulated with the respective proteins for 24 h and the number of cytokine producing cells per well was determined (FIG. 6). Stimulation with the two allergen derivatives induced the highest number of IFN-γ and IL-5 in the homologous situation. However, immunisation with the derivatives also stimulated cytokine production after stimulation with the heterologous derivatives and a modest reaction with the wild-type allergen, indicating T cell-crossreactive properties for all three molecules.

1. Method for identifying a hypoallergenic derivative of a wild-type allergen comprising:
determining a three-dimensional structure of a wild-type allergen,
introducing at least one point mutation into said wild-type allergen, thereby obtaining a mutated allergen,
identifying a hypoallergenic derivative of said wild-type allergen by detecting a destabilization or change of the three-dimensional structure of the mutated allergen compared to the wild-type allergen by determining energy differences between the wild-type allergen and the mutant allergen expressed as a Z-score.

2. Method according to claim 1, wherein the three-dimensional structure is determined by a method selected from the group consisting of nuclear magnetic resonance (NMR) spectroscopy, X-ray crystallography, computational methods, circular dichroism and combinations thereof.

3. Method according to claim 2, wherein the three-dimensional structure is determined via circular dichroism, especially by a combination of methods comprising circular dichroism.

4. Method according to claim 1, wherein the mutated allergen is further subjected to an IgE binding assay or a mediator release assay.

5. Method according to claim 4, wherein the IgE binding assay is a RIST (radio immunosorbens test), a RAST (radio allergo-sorbens test) or a Western blot.

6. Method according to claim 4, wherein the mediator release assay is a CAST (cellular allergen stimulation test), a histamine release assay, a leukotriene C4 release assay, a cysteinyl leukotriene release assay, tryptase assay or rat basophil leukemia (RBL) cell release assay.

7. Method according to claim 1, wherein the wild-type allergen is selected from the group consisting of Amb a 1, Art v 1, Par j 1, Cyn d 1, Dac g 1, Lol p 1, Phl p 1, Phl p 2, Phl p 4, Phl p 5, Phl p 6, Aln g 1, Bet v 1, Cas s 1, Gar a 1, Que a 1, Ole e 1, Cry j 1, Jun a 1, Der f 1, Der m 1, Der p 1, Equ c 1, Fel d 1, Alt a 1, Cla h 2, Asp f 1, Pen b 13, Cand a 1, Api m 1, Pol a 1, Vesp c 1; Bra j 1, Bra n 1, Bra o 3, Bra r 1, Zea m 14, Api g 1, Dau c 1, Mal d 1, Pru ar 1, Pru av 1, Ara h 1, Cap a 1w, Lyc e 1, Hev b 1, Hev b 2, Hev b 3, Hev b 4, Hev b 5, Hev b 6 and Hom s 1.

8. Hypoallergenic derivative of a wild-type allergen exhibiting a three-dimensional structure having a Z-score, which differs from a Z-score of the three-dimensional structure of the wild-type allergen, and being derived from an allergen selected from the group consisting of Amb a 1, Art v 1, Par j 1, Cyn d 1, Dac g 1, Lol p 1, Phl p 1, Phl p 2, Phl p 4, Phl p 5, Phl p 6, Aln g 1, Bet v 1, Cas s 1, Cor a 1, Que a 1, Ole e 1, Cry j 1, Jun a 1, Der f 1, Der m 1, Der p 1, Equ c 1, Fel d 1, Alt a 1, Cla h 2, Asp f 1, Pen b 13, Cand a 1, Api m 1, Pol a 1, Vesp c 1, Bra j 1, Bra n 1, Bra o 3, Bra r 1, Zea m 14, Api g 1, Dau c 1, Mal d 1, Pru ar 1, Pru av 1, Ara h 1, Cap a 1w, Lyc e 1, Hev b 1, Hev b 2, Hev b 3, Hev b 4, Hev b 5, Hev b 6 and Hom s 1.

9. Hypoallergenic derivative according to claim 8, wherein the allergen derivative comprises at least one mutation compared to the wild-type allergen.

10. Hypoallergenic derivative according to claim 8, wherein the at least one mutation is selected from the group consisting of amino acid exchange, amino acid deletion and amino acid insertion.

11. Hypoallergenic derivative according claim 8, wherein the allergen is Bet v 1.

12. Hypoallergenic derivative according to claim 11, wherein the derivative is selected from the group consisting of SEQ ID No. 2, SEQ ID No. 3 and SEQ ID No. 4.

13. Vaccine formulation comprising a hypoallergenic derivative of a wild-type allergen that contains at least one point mutation resulting in a mutated allergen, wherein said hypoallergenic derivative of said wild-type allergen contains a destabilized or changed three-dimensional structure compared to the wild-type allergen reflected in enemy differences between the wild-type allergen and the mutated allergen expressed as a Z-score.

14. Method of claim 1, wherein the hypoallergenic derivative is further manufactured into a vaccine for preventing and treating allergies.

15. Vaccine formulation comprising at least one of the hypoallergenic derivatives from the allergens of claim 8.


SEQUENCE LISTING (21 sequences)

SEQ ID NO: 1 - protein, 159 aa, Artificial Sequence
  recombinant wild type Bet v1a
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRAVESYLLAHSDAYN

SEQ ID NO: 2 - protein, 159 aa, Artificial Sequence
  recombinant Bet Mut 123
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKKSNEIKIVATPDGGSIKKISNKYHTKGDHEEKAEQVKASKEMGETLLRAVESYLLAHSDAYN

SEQ ID NO: 3 - protein, 159 aa, Artificial Sequence
  recombinant Bet Mut 1234
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKKSNEIKIVATPDGGSIKKISNKYHTKGDHEEKAEQVKASKEMGETLLRPVESYLLAHSDAYN

SEQ ID NO: 4 - protein, 159 aa, Artificial Sequence
  recombinant Bet Mut 4
GVFNYETETTSVIPAARLFKAFILDGDNLFPKVAPQAISSVENIEGNGGPGTIKKISFPEGFPFKYVKDRVDEVDHTNFKYNYSVIEGGPIGDTLEKISNEIKIVATPDGGSILKISNKYHTKGDHEVKAEQVKASKEMGETLLRPVESYLLAHSDAYN

SEQ ID NO: 5 - DNA, 30 bp, Artificial Sequence
  forward primer
caccgaattcatgggtgttttcaattacga

SEQ ID NO: 6 - DNA, 109 bp, Artificial Sequence
  reverse primer
cgactctagacatggtcacctttggtgtggtacttgttgctgatcttcttgatggatcctccatcaggggttgccactatctttatctcgttggacttcttctccaatg

SEQ ID NO: 7 - DNA, 128 bp, Artificial Sequence
  reverse primer
cgactctagattagttgtaggcatcggagtgtgccaagaggtagctctcaactggcctcaaaagtgtctcgcccatttctttacttgccttaacctgctctgccttctcctcatggtcacctttggtg

SEQ ID NO: 8 - DNA, 24 bp, Artificial Sequence
  forward primer
caccatgggtgttttcaattacga

SEQ ID NO: 9 - DNA, 30 bp, Artificial Sequence
  reverse primer
gatcgaattcttagttgtaggcatcggagt

SEQ ID NO: 10 - DNA, 24 bp, Artificial Sequence
  forward primer
attggagaagaagtccaacgagat

SEQ ID NO: 11 - DNA, 31 bp, Artificial Sequence
  reverse primer
gatctctagattagttgtaggcatcggagtg

SEQ ID NO: 12 - DNA, 30 bp, Artificial Sequence; identical to SEQ ID NO: 5
  forward primer

SEQ ID NO: 13 - DNA, 24 bp, Artificial Sequence
  reverse primer
atctcgttggacttcttctccaat

SEQ ID NO: 14 - DNA, 31 bp, Artificial Sequence; identical to SEQ ID NO: 11
  reverse primer

SEQ ID NO: 15 - DNA, 22 bp, Artificial Sequence
  forward primer
aggatccatcaagaagatcagc

SEQ ID NO: 16 - DNA, 31 bp, Artificial Sequence; identical to SEQ ID NO: 11
  reverse primer

SEQ ID NO: 17 - DNA, 22 bp, Artificial Sequence
  reverse primer
gctgatcttcttgatggatcct

SEQ ID NO: 18 - DNA, 31 bp, Artificial Sequence; identical to SEQ ID NO: 11
  reverse primer

SEQ ID NO: 19 - DNA, 22 bp, Artificial Sequence
  forward primer
tgaccatgaggagaaggcagag

SEQ ID NO: 20 - DNA, 22 bp, Artificial Sequence
  reverse primer
ctctgccttctcctcatggtca

SEQ ID NO: 21 - DNA, 30 bp, Artificial Sequence; identical to SEQ ID NO: 9
  reverse primer